(19) United States
(12) Patent Application Publication
(10) Pub. No.: US 2003/0139296 A1
(43) Pub. Date: Jul. 24, 2003

(54) Use of vitamin b1 as agents for controlling plant diseases

(76) Inventors: Yong-Hwan Lee, Kyunggi-do (KR); Il-Pyung Ahn, Kyunggi-do (KR); Nam-Soo Jwa, Kyunggi-do (KR); Soon-Ok Kim, Kyunggi-do (KR); Chan-Ho Park, Kyunggi-do (KR); Sook-Young Park, Jeollanam-do (KR); Young-Joon Son, Busan (KR); Dal-Soo Kim, Taejon (KR); Sam-Jae Chun, Taejon (KR); Yeon-Mi Lee, Taejon (KR); Yun-Kyung Cho, Jeollabuk-do (KR); Cheol-Yong Bae, Kyungsangnam-do (KR)

Correspondence Address: BIRCH STEWART KOLASCH & BIRCH, PO BOX 747, FALLS CHURCH, VA, 22040-0747, US

(21) Appl. No.: 10/239,574

(22) PCT Filed: Mar. 24, 2001
(86) PCT No.: PCT/KR01/00478

(30) Foreign Application Priority Data
Mar. 24, 2000 (KR) 2000/15155

(51) Int. Cl. A61K 31/51; A01N 43/54; A01N 43/78

(52) U.S. Cl. 504/239; 514/276

(57) ABSTRACT

The present invention relates to an agent for controlling plant diseases containing vitamin B1, or salts or derivatives thereof as an active ingredient, which exhibits excellent disease controlling effects by rapid induction of defense-related genes in plants infected with pathogens.

### TECHNICAL FIELD

The present invention relates to a use of vitamin B1 as an agent for controlling plant diseases, and more specifically to a use of vitamin B1, its salts or derivatives thereof as an agent for effectively controlling plant diseases, which induces to express defense-related genes in the early stages of infection by plant pathogens,

### BACKGROUND ART

The increased yield of crops has recently been desired with the explosive increase in the poppulation. Thus, a demand for controlling plant diseases against plant pathogens which are threat to the stable crop production has been on the rise. Agricultural pesticides and chemical fertilizers which have been used from the beginning of the 20th century are indispensable for the stable production of crops, but have lead to consumer's distrusts about food safety and have negatively influenced on the environment. Furthermore, the appearance of pathogenic strains resistant to the agricultural chemicals became another serious problem. Therefore, many studies have been concentrated on developing a novel method for controlling plant diseases safely and efficiently. Particularly, the method for controlling plant diseases by enhancing a plant's defense mechanisms has occupied an important position.

The plants show very sophisticated defense mechanisms against the invasion of pathogens. It is known that such defense mechanisms include hypersensitive reaction (HR), synthesis of phytoalexin, reinforcement of cell walls, and formations of enzymes degrading cell walls of microorganisms and proteinase inhibitors.

Recent studies about the defense mechanisms due to the advancement of genetic engineering have been progressed. As a result, it has been found that most of the defense mechanisms are accompanied with the expression of defense-related genes, such as PR1 (pathogenesis-related gene 1), PBZ1 (probenazole-inducible gene 1), and POX22.3 (peroxidase gene 22.3) of rice, and PAL (phenylalanine ammonia lyase), APX (ascorbate peroxidase) and HMGR (3-hydroxy-3-methyl-glutaryl-CoA reductase) of tomato, etc.

Therefore, it is vigorously in progress to increase plant resistance against pathogens by artificially inducing the expression of the defense genes. It has been reported that, for example, benzothiadiazole induces systemic acquired resistance (SAR) in Solanaceae or Cruciferaceae plants (Lawton, et al., 1996), and probenazole induces SAR of rice, thereby increasing resistance to the plant pathogens (Midoh and Iwata, 1996). However, the above agents have problems that they required a long period of time in expressing defense gene after the treatment, as well as did not sufficiently exhibit persistent activity against the plant pathogens. Therefore, the development of novel agents which can effectively control plant diseases by inducing the expression of defense genes in plants from the early stage of infection has been desired keenly.

### DETAILED DESCRIPTION OF THE INVENTION

Under these circumstances, the present inventors have engaged in continuous studies about a novel agent to artificially induce the expression of the defense genes in plants, and as the result, found a new fact that vitamin B1, which is harmless to human or animal and plays a role in increasing immunity and resistance to diseases in human and animal, also inhibits various plant diseases without being restricted only to some specific diseases. Through the above fact, the present inventors thought that vitamin B1 induces the expression of defense genes in plants rather than inhibits plant pathogens, and finally confirmed the expression of the defense genes by vitamin B1 through repetitious experiments and completed the present invention.

Therefore, it is an object of the present invention to provide a composition for controlling plant diseases, which is nontoxic to human, animal, plant and environment, and which effectively controls plant diseases against plant pathogens by inducing the expression of defense genes in plants.

It is another object of the present invention to provide a method for controlling plant diseases using the above composition.

In order to accomplish the above objects, the present invention provides a composition for controlling plant diseases comprising vitamin B1, its salts or derivatives thereof as an effective ingredient.

The present invention also relates to a method for controlling plant diseases comprising applying the above composition to the plant, its seed or its habitat.

Hereinafter, the present invention is explained in detail.

The plant herein includes monocotyledonous plants, dicotyledonous plants or seeds thereof Further, the plant diseases include rice blast, late blight, powdery mildew, rust of various crops and bacterial leaf blight of rice, but are not limited to them.

Salts and derivatives of vitamin B1 to be used in the present invention include all the salts and derivatives of vitamin B1 commonly used in the technical field, but are not limited to a specific kind. For example, as salts of vitamin B1 are included hydrochloride and mononitrate, and as derivatives of vitamin B1 are included monophosphate chloride and pyrophosphate chloride, but are not limited to them.

The content of the effective ingredient used in the present invention can be appropriately adjusted depending upon type of plants to be treated, kind and condition of plant diseases, and time and method to be treated, but preferably within the concentration of 5 mM to 100 mM.

In the present invention, vitamin B1, its salts or derivatives thereof may be used alone in the form of solution in distilled water, or formulated with other adjuvants. For example, vitamin B1, or salts or derivatives thereof may be formulated into wetting agent with conventional adjuvants such as sodium dodecyl sulfate (SDS), sodium lignin sulfonate (SLS) and kaolin.

In addition, the compositions according to the present invention may be mixed with other conventional synthetic pesticides, biological pesticides or fertilizers.

The composition according to the present invention can be used similarity with the application volume of conventional agricultural chemicals. For example, the liquid preparation can be sprayed in the amount enough to uniformly drip wet the surface of the plants to be treated. It is confirmed that the composition according to the present invention can be treated even at any growth stages from seed to mature plants. Further, it is confirmed that the compositions according to the present invention do not influence on germination or growth of plants at the concentration of not more than 100 mM in any time at all, and do not any chemical damages to plants when visually observed after 10 days from the treatment. The agricultural composition of the present invention can be treated with any customary method such as spraying, pouring, soaking, etc.

On the other hand, examples of vitamins effective for controlling plant disease by inducing the expression of defense genes in plants include vitamin B6 and vitamin C in addition to vitamin B1. Among them, it is confirmed that vitamin B1 is the most effective for inducing the expression of defense genes in plants. Therefore, agents according to the present invention can further contain vitamin B6 and/or vitamin C together with vitamin B1, its salts or derivatives thereof.

In order to identify the controlling efficacy in the present invention, the incidence of lesion and progression thereof are investigated in accordance with the following steps:

dissolving each hydrochloride, mononitrate, monophosphate chloride and pyrophosphate chloride of vitamin B1 in distilled water, spray-treating on rice, tomato, barley and wheat with the solution thus obtained, or soak-treating the rice seeds in the solution thus obtained, and inoculating with plant pathogenic fungi and bacteria, e.g. Magnaporthe grisea KJ201, Phytophthora infestans PIT, Erysiphe graminis and Puccina recondita, and Xanthomonas oryzae pv. oryzae KX021, respectively.

Further, it is identified the expression of defense genes in RNA obtained by spray-treating on rice with an aqueous vitamin B1 hydrochloride solution, and/or inoculating with plant pathogens such as Xanthomonas oryzae pv. oryzae KX021, and extracting total RNA therefrom. At this time, PR1, PBZ1 and POX22.3 genes are used as probes. Furthermore, it is identified the expression of defense genes in RNAs obtained from soak-treating tomato in an aqueous vitamin B1 hydrochloride solution. At this time, PAL, APX and HMGR genes are used as probes.

Magnaporthe grisea KJ201 and Xanthomonas oryzae pv. oryzae KX021 used in the present invention are made available from National Institute of Agricultural Science and Technology, Rural Development Administration (Seodun-Dong, Suwon, Kyonggi-Do, Korea). Phytophihora infestans PIT was isolated from infected potato at Daejeon, Korea, and Erysiphe graminis and Puccina recondita are made available from Korea Research Institute of Chemical Technology at Daejeon, Korea.

### BRIEF DESCRIPTION OF THE DRAWINGS

FIG. 1 is a X-ray film photograph showing the expression of defense genes when rice was treated with vitamin B1 hydrochloride and/or infected with rice blast fungus.

FIG. 2 is a X-ray film photograph showing the expression of defense genes when rice was treated with vitamin B1 hydrochloride at different time intervals.

FIG. 3 is a X-ray film photograph showing the expression of defense genes when rice was treated with vitamin B1 hydrochloride and/or infected with bacterial leaf blight bacterium.

FIG. 4 is a X-ray film photograph showing the expression of defense genes in vitamin B1 hydrochloride-treated tomato with time intervals.

### BEST MODE FOR CARRYING OUT THE INVENTION

Hereinafter, the present invention is more specifically illustrated by the following examples. However, these examples do not intend to limit the present invention in any manner.

### EXAMPLE 1

### Preparation of Vitamin B1 Hydrochloride Wettable Powder

300 g of vitamin B1 hydrochloride (thiamine HCl; Cat. No. T4625; Sigma Co.), and 20 g of sodium dodecyl sulfate (SDS; Cat. No. 192-08675, Wako Co.), 30 g of sodium lignin sulfonate (SLS; Cat. No 47103-8; Aldrich Co.) and 650 g of kaolin (Cat. No. 117-00025, Wako Co.) as adjuvants were uniformly mixed in a vinyl bag. Subsequently, the mixture was ground to obtain 1 kg of the title wettable powder.

### EXAMPLE 2

### Preparation of Vitamin B1 Mononitrate Wettable Powder

1 kg of the title wettable powder was obtained according to the same procedure as Example 1, except that vitamin B1 mononitrate (thiamine mononitrate; Cat. No. T1054; Spectrum Quality Products. Inc.) was used instead of vitamin B1 hydrochloride.

### EXAMPLE 3

### Preparation of Vitamin B1 Monophosphate Chloride Wettable Powder

1 kg of the title wettable powder was obtained according to the same procedure as Example 1, except that vitamin B1 monophosphate chloride (thiamine monophosphate chloride; Cat. No. T8637; Sigma Co.) was used instead of vitamin B1 hydrochloride.

### EXAMPLE 4

### Preparation of Vitamin B1 Pyrophosphate Chloride Wettable Powder

1 kg of the title wettable powder was obtained according to the same procedure as Example 1, except that vitamin B1 pyrophosphate chloride (thiamine pyrophosphate chloride; Cat. No. T0996 1; Fluka) was used instead of vitamin B1 hydrochloride.

The components and contents of the wettable powders prepared from Examples 1 to 4 are shown in Table 1 below.

TABLE 1

| Component | | Exa. 1 | Exa. 2 | Exa. 3 | Exa. 4 |
|---|---|---|---|---|---|
| Effective | Vitamin B1 hydrochloride | 30 | — | — | — |
| ingredient | Vitamin B1 mononitrate | — | 30 | — | — |
| | Vitamin B1 monophosphate chloride | — | — | 30 | — |
| | Vitamin B1 pyrophosphate chloride | — | — | — | 30 |
| Adjuvant | SDS | 2 | 2 | 2 | 2 |
| | SLS | 3 | 3 | 3 | 3 |
| | Kaolin | 65 | 65 | 65 | 65 |

### TEST EXAMPLE 1

### Expression Analyses of Defense Genes in Rice Against Rice Blast

Rice seeds(cultivar Hwacheong) were soaked in Homai wettable powder for seed-disinfection for 1 day and then stimulated germination at 28° C. for 2 days. After the seeds were planted in the bed soil for agriculture and cultivated to its fourth-leaf stage in a greenhouse, the rice seedlings were used in the following experiments.

The strain Magnaporthe grisea KJ201 causing rice blast was incubated in oat agar medium at 25° C. for 12-15 days under continuous light condition to form conidia. Tween 80 (polyoxyethylene glycol) at the concentration of 250 ppm (v/v) was added in the medium, and then the spores were harvested. The harvested spores were titrated using hemacytometer to bring the final concentration to 5×10⁵ spores/ml and spore suspension was spray-inoculated on rice leaves and stems in 20 rice pots, respectively, in the amount of 5 ml per pot. After inoculating, the pots were left at room temperature for 20 minutes, dried so as not to drip wet, and then left at 25° C. and 100% of relative humidity for 24 hours under dark condition (Group A: rice inoculated with rice blast fungi only).

On the other hand, vitamin B1 hydrochloride at the final concentration of 50 mM, and Tween 80 at the final conhcentration of 250 ppm were dissolved in distilled water. The solution thus prepared was spray-treated on rice leaves and stems in 20 rice pots, respectively, in the amount of 5 ml per pot (Group B: rice treated with vitamin B1 hydrochloride only).

Further, vitamin B1 hydrochloride was spray-treated on 20 rice pots as in Group B. 24 hours after spray-treating, rice blast fungus was inoculated as in Group A (Group C: rice treated with vitamin B1 hydrochloride and then inoculated with rice blast fungus).

After rice blast fungus was inoculated, groups A, Band C were placed in the growth chamber at 25° C. and 80% relative humidity while light and darkness were changed at an interval of 12 hours. Rice leaves of each group were harvested at an interval of 24 hours from 0 to 96 hours after the inoculation. The harvested rice leaves were immersed in liquid nitrogen, quick-freezed, and then stored in a freezer. After the harvested rice leaves were finely ground in mortar and pestle with the addition of liquid nitrogen thereto, total RNAs were extracted according to the conventional extraction methods. The concentrations of RNA were quantified by measuring each absorbance in the wavelength of 260 nm. Subsequently, the RNAs were subjected to electrophoresis in an amount of 15 μg, respectively, and then transferred into a nylon membrane using 10×SSC. In order to employ PR1, PBZ1 and POX22.3 (full-length genes having sequences registered in NCBI GenBank), which are used as indices for the expression of defense gene in rice, as probes, the genes were radioactively labeled and carried out a hybridization at 42° C. for 16 hours under 20 rpm. The nylon membrane was treated with a washing buffer containing 0.2% sarcosine(2×SSC), and subsequently exposed to X-ray film to measure the expression degree of the probes.

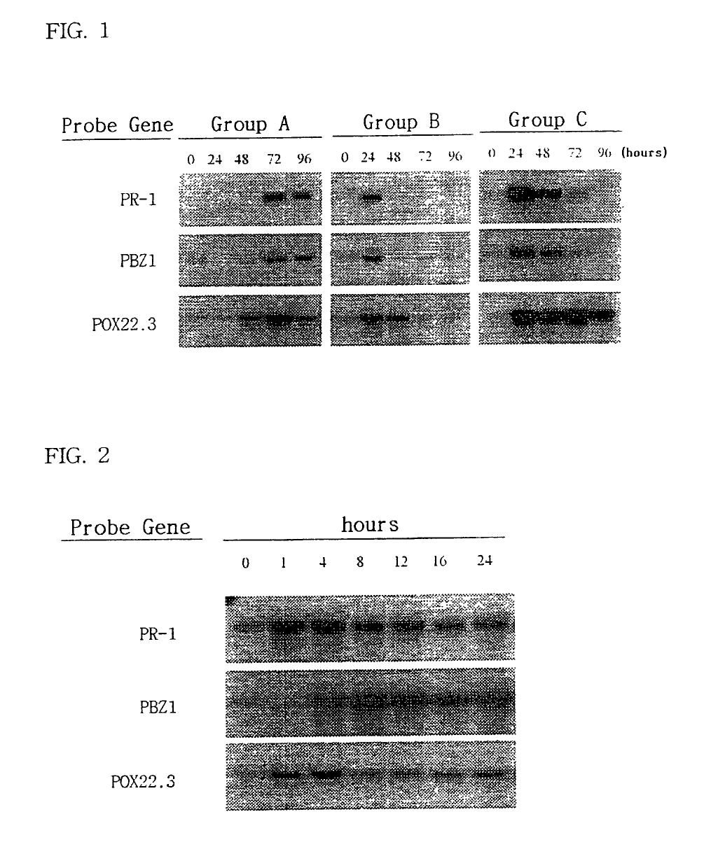

The results are shown in FIG. 1. As shown in FIG. 1, the expression of defense genes was detected 72 hours after inoculation in rice inoculated with rice blast fungus only (group A); whereas their expressions were detected after 24 hours in the rice treated with vitamin B1 hydrochloride only (group B). In addition, the expression of all three defense genes were detected 24 hours after inoculation in rice treated with vitamin B1 hydrochloride and then inoculated with rice blast fungus (group C). Furthermore, their expressions were considerably increased, compared to groups A and B.

### TEST EXAMPLE 2

### Expression Analysis of Defense Genes in Rice

Rice plant was treated with vitamin B1 hydrochloride according to the same procedure as Test Example 1. The rice leaves were harvested 0, 1, 4, 8, 12, 16 and 24 hours after treatment, respectively. The expression of defense genes in rice was measured according to the same method as Test Example 1.

The results are shown in FIG. 2. 4 hours after the treatment, and all defense genes tested were induced to express 4 hours after treatment in rice treated with vitamin B1 hydrochloride only.

### TEST EXAMPLE 3

### Expression Analysis of the Defense Genes in Rice Against Bacterial Leaf Blight

Rice(cultivar Hwacheong) was soaked in Homai wettable powder for seed-disinfection for 1 day and then stimulated germination at 28° C. for 2 days. After the seeds were planted in the bed soil and cultivated to its fourth-leaf stage in a greenhouse, the rice seedlings were used in the following experiments.

The strain Xanthomonas oryzae pv. oryzae KX021 causing bacterial leaf blight was incubated in polypeptone-sucrose agar medium at 30° C. for 72 hours under dark condition. Polyoxyethylene glycol (Tween 80) solution at the concentration of 250 ppm(v/v) was added to the medium and then the bacterial inoculum was harvested. After the inoculum was titrated so as to be 0.5 of absorbance in the wavelength of 600 nm by spectrophotometer, the bacterial solution was scissors inoculated on 20 rice pots. The rice pots were left at 25° C. and 100% of relative humidity for 24 hours under dark condition (Group A: rice inoculated with leaf blight bacteria only).

On the other hand, a solution containing vitamin B1 hydrochloride at the final concentration of 50 mM in distilled water and Tween 80 at the final concentration of 250 ppm as a spreader was prepared. The solution thus prepared was spray-treated on rice leaves and stems in 20 rice pots, respectively, in the amount of 5 ml per pot using atomizer (Group B: rice treated with vitamin B1 hydrochloride only).

In addition, vitamin B1 hydrochloride was spray-treated on 20 rice pots as in Group B. 24 hours after treating, rice leaf blight bacteria were inoculated as in Group A (Group C: rice treated with vitamin B1 hydrochloride and then inoculated with leaf blight bacteria). The expressions of defense genes in each group were measured using the probes, according to the same procedure as Test Example 1.

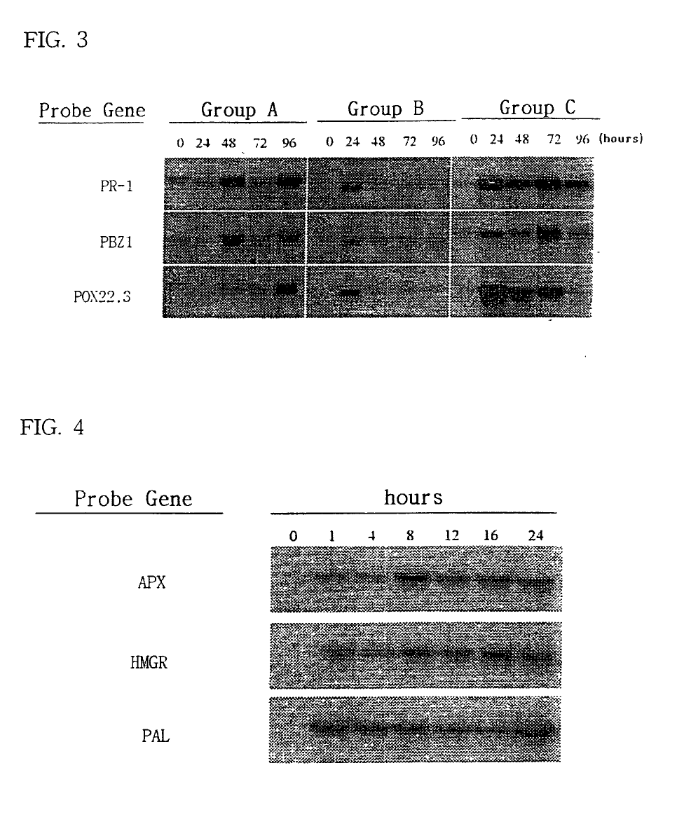

The results are shown in FIG. 3. As shown in FIG. 3, the expressions of defense genes tested were detected 48 hours after inoculation in rice inoculated with leaf blight bacteria only (group A); whereas their expressions were detected 24 hours after treatment in the vice treated with vitamin B1 hydrochloride only (group B). In addition, expressions of all three defense genes were detected 24 hours after inoculation in the rice treated with vitamin B1 hydrochloride and then inoculated with leaf blight bacteria (group C). Furthermore, their expressions were considerably increased, compared to groups A and B.

### TEST EXAMPLE 4

### Expression Analysis of Defense Genes in Tomato

Tomato(cultivar Sekwang; HeungNong Seeds and Saplings Co, var. reg. No: VT-Hy43) was planted in the bed soil for horticulture Bunong, cultivated in a greenhouse, and then used in the following experiments. Tomato plants having the specific size were selected, soils attached to the roots were removed, and then cut the main root with a razor blade. 200 ml of aqueous vitamin B1 hydrochloride solution at the concentration of 50 u uM was poured into 250 ml beaker and the opening of beaker was covered with aluminum foil. The foil was made with a hole and the tomato stem was soaked therein. 1, 4, 8, 12, 16 and 24 hours after soaking, the expression degrees of PAL, APX and HMGR (available from Korea Research Institute of Bioscience and Biotechnology), which are used as indices for the expression of defense gene in tomato, were examined, according to the same procedure as Test Example 1.

The results are shown in FIG. 4. As shown in FIG. 4, all the defense genes were expressed in tomatoes treated with vitamin B1 hydrochloride only within 1 hour.

### TEST EXAMPLE 5

### Test on Control Efficacy Against Rice Blast Disease

(A) Aqueous solutions containing each vitamin B1 hydrochloride at the final concentrations of 5, 10, 20, 50 and 100 mM with Tween 80 at the final concentration of 250 ppm, an an aqueous solution containing Tween 80 at the final concentration of 250 ppmonly were prepared. Each solution thus prepared was spray-treated on rice leaves and stems in 20 rice pots, respectively, in the amount of 5 ml per pot according to the same procedure as Test Example 1. 4 hours after the treatment, spore suspension of rice blast fungi was spray-inoculated on rice according to the same procedure as Test Example 1. After inoculating, the pots were left at room temperature for 20 minutes, dried till the inocula were not drip wet, and then left at 25° C. and 100% of relative humidity for 24 hours under dark condition. The disease progress was investigated in the growth chamber at 25° C. and 80% relative humidity while light and darkness were changed at an interval of 12 hours with time. 1 week after the inoculation, the degree of the disease severity was examined based upon the lesion type, lesion size and the ratio of diseased area. The results are shown in Table 2 (mean value±standard deviation).

1) L.T. (lesion type): 0˜4

0=no changed

1=fine brown spots

2=1˜2 mm long brown spots, about 1 mm long brown spots having light gray center, or yellow spots having about 1˜2 mm long brown center

3=round spots having gray center, or 1˜2 mm long gray spots

4=diamond-shaped brown spots having not less than 1 cm long gray center

2) D.S.(Disease Severity): 0˜10

0=no symptoms of disease;

1=L.T. 1 of less than 50% among the total leaves

2=L.T. 1 of not less than 50% among the total leaves

3=developed to L.T. 2

4=appearance of L.T. 3

5=L.T. 3 of not less than 50% among the total leaves

6=appearance of gray spots corresponding to L.T.3

7=developed to L.T. 4

8=L.T. 4 occupied the most parts of leaves

9=leaves start to die

10=dead leaves of not less than 50% among the total leaves

TABLE 2

| | Degree of the | Control |
|---|---|---|
| Treated preparation (concentration treated) | disease severity | value(%) |
| Vitamin B1 HCl(5 mM) + Tween 80 | 6.5 ± 0.6 | 21.2 ± 7.2 |
| (250 ppm) | | |
| Vitamin B1 HCl(10 mM) + Tween 80 | 5.8 ± 1.5 | 30.3 ± 18.2 |
| (250 ppm) | | |
| Vitamin B1 HCl(20 mM) + Tween 80 | 2.3 ± 1.4 | 72.7 ± 17.0 |
| (250 ppm) | | |
| Vitamin B1 HCl(50 mM) + Tween 80 | 2.8 ± 1.5 | 66.7 ± 18.2 |
| (250 ppm) | | |
| Vitamin B1 HCl(100 mM) + Tween 80 | 1.1 ± 0.8 | 87.3 ± 9.7 |
| (250 ppm) | | |
| Tween 80(250 ppm) | 8.3 ± 0.5 | — |

[Control value(%) = {(a degree of the disease severity in the Tween 80-treated rice - a degree of the disease severity in the vitamin B1 HCl and Tween 80-treated rice)/a degree of the disease severity in the Tween 80-treated rice} × 100]

As shown in Table 2, when treated with vitamin B1 hydrochloride at the concentrations of 5 and 10 mM, the control values were relatively low (not more than 30%). However, when treated with vitamin B1 hydrochloride at the concentration of not less than 20 mM, the control values were high and maximum control efficacy was about 87% with 100 mM treatment.

(B) The degree of the disease severity was examined according to the same method as the above (A), except that wettable powders prepared from Examples (1)˜(4) were prepared to the concentrations of 30 and 50 mM, respectively, based upon the respective effective ingredients.

The results are shown in Table 3 (mean value±standard deviation)

TABLE 3

| | Concentration | | |
|---|---|---|---|
| Treated | treated | Degree of the disease | |
| preparation | (mM) | severity | Control value (%) |
| Exa. 1 | 30 | 2.0 ± 0.5 | 80.3 |
| | 50 | 1.4 ± 0.3 | 85.7 |
| Exa. 2 | 30 | 1.8 ± 0.2 | 82.3 |
| | 50 | 1.3 ± 0.1 | 87.3 |
| Exa. 3 | 30 | 1.3 ± 0.2 | 82.3 |
| | 50 | 1.0 ± 0.3 | 90.3 |
| Exa. 4 | 30 | 2.0 ± 0.4 | 80.7 |
| | 50 | 1.6 ± 0.5 | 84.3 |

| | | |
|---|---|---|
| Non-treated rice | 10.0 ± 0.0 | — |

[Control value(%) = {(a degree of the disease severity in the non-treated rice - a degree of the disease severity in the preparations of Examples-treated rice)/a degree of the disease severity in the non-treated rice} × 100]

As shown in Table 3, when treated with the preparations of Examples 1˜4 at the concentration of not less than 30 mM, the control values were high (not less than 80%).

### TEST EXAMPLE 6

### Controlling Effect Test on Bacterial Leaf Blight in Rice

An aqueous solution containing vitamin B1 hydrochloride at each final concentration of 5, 10, 20, 50 and 100 mM and Tween 80 at final concentration of 250 ppm, and an aqueous solution containing Tween 80 at the concentration of 250 ppm only were prepared. Each solution thus prepared was spray-treated on rice leaves and stems in 20 rice pots, respectively, in the amount of 5 ml per pot according to the same procedure as Test Example 1. 4 hours after the treatment, leaf blight bacteria were scissors-inoculated on rice according to the same procedure as Test Example 3. After inoculating, the pots were left at 25° C. and 100% of relative humidity for 24 hours under dark condition The progression of the disease was investigated in the growth chamber at 25° C. and 80% relative humidity while light and darkness were changed at an interval of 12 hours with time. 1 week after the inoculation, the degree of the disease severity was examined by measuring the distance from the inoculated points to the symptoms of disease The results are shown in Table 4 (mean value±standard deviation)

TABLE 4

| | Degree of the | Control |
|---|---|---|
| Treated preparation (concentration treated) | disease severity | value(%) |
| Vitamin B1 HCl(5 mM) + Tween 80 | 8.7 ± 0.6 | 10.3 ± 6.2 |
| (250 ppm) | | |
| Vitamin B1 HCl(10 mM) + Tween 80 | 4.2 ± 1.4 | 56.3 ± 14.4 |
| (250 ppm) | | |
| Vitamin B1 HCl(20 mM) + Tween 80 | 3 1 ± 2.5 | 68.3 ± 25.9 |
| (250 ppm) | | |
| Vitamin B1 HCl(50 mM) + Tween 80 | 0.2 ± 0.3 | 97.6 ± 3.1 |
| (250 ppm) | | |
| Vitamin B1 HCl(100 mM) + Tween 80 | 0.3 ± 0.1 | 97.2 ± 1.0 |
| (250 ppm) | | |
| Tween 80(250 ppm) | 9 7 ± 1.2 | — |

[Control value (%) is calculated by the formula as described in Table 2.]

As shown in Table 4, when treated with vitamin B1 hydrochloride at the concentration of 5 mM, the control value was low (about 30%). However, when treated with vitamin B1 hydrochloride at the concentrations of 10, 20, 50 and 100 mM, the control values were high (not less than 56%, 68%, 97% and 97%, respectively).

### TEST EXAMPLE7

### Controlling Effect Test on Rice Blast by Seed-soaking

Rice(cultivar Hwacheong) was soaked in Homai wettable powder for seed-disinfection for 1 day and then stimulated germination at 28° C. for 2 days The stimulated seeds were soaked in vitamin B1 hydrochloride solution and distilled water at the concentrations of 20 mM and 50 mM, respectively, at 28° C. for 1 day, and then planted in the bed soil for agriculture. The seed-treated rice was cultivated to its fourth-leaf stage in a greenhouse.

Spore suspension of rice blast fungi was obtained according to the same procedure as Test Example 1, and then spray-inoculated on rice leaves and stems in 20 rice pots, respectively, in the amount of 5 ml per pot. After inoculating, the pots were left at room temperature for 20 minutes, dried till the inocula were not drip wet, and then left at 25° C. and 100% of relative humidity for 24 hours under dark condition. The progression of disease was investigated in the growth chamber at 25° C. and 80% relative humidity while light and darkness were changed at an interval of 12 hours with time. The degree of the disease severity was examined according to the same method as Test Example 5(A). The results are shown in Table 5(mean value±standard deviation).

TABLE 5

| Treated | Concentration | Degree of the disease | |
|---|---|---|---|
| preparation | treated | severity | Control value (%) |
| Vitamin B1 | 20 mM | 5.4 ± 3.2 | 44.4 ± 32.8 |
| HCl | | | |
| | 50 mM | 2.3 ± 1.9 | 76.8 ± 19.5 |
| Distilled | — | 9.8 ± 1.7 | — |
| water | | | |

[Control value(%) = {(a degree of the disease severity in the distilled water-treated rice - a degree of the disease severity in the vitamin B1 HCl-treated rice)/a degree of the disease severity in the distilled water-treated rice} × 100]

As shown in Table 5, when treated with vitamin B1 hydrochloride at the concentrations of 20 and 50 mM at the time of germination, the control values were 44% and 77%, respectively.

### TEST EXAMPLE 8

### Controlling effect Test on Tomato Late Blight

Tomato(cultivar Sekwang; HeungNong Seeds and Saplings Co, var. reg. No: VT-Hy-43) was planted in the bed soil for horticulture(Bunong Co), cultivated in a greenhouse for 4 weeks, and then used in the following experiments. Wettable powders prepared from Examples (1)˜(4) were prepared to the concentrations of 30 and 50 mM, respectively. based upon the respective effective ingredients, and then spray-treated on tomato leaves and stems using atomizer, respectively, in the amount of 5 ml per pot.

On the other hand, the strain Phytophthora infestans PIT causing tomato late blight was incubated in Rye B agar medium at 20° C. for 7-10days under light condition to form sporangia. 15 ml of distilled water per petri dish was added thereto, and then sporangia were harvested using a sterilized brush. The harvested sporangia were left at 4° C. for 3 hours to obtain zoospores. The concentration of the zoospores was titrated using hemacytometer to bring the final concentration to 5×10⁴ zoospores/ml. 4 hours after spray-treating, the titrated zoospores were spray-inoculated on tomatoes in the amount of 5 ml per pot. The progression of the disease was investigated at 20° C. and 100% relative humidity with time. 4 days after the inoculation, the degree of the disease severity was examined based upon the lesion type, lesion size and the ratio of diseased area (see. Test Example 5(A)). The results are shown in Table 6 (mean value±standard deviation).

TABLE 6

| | Concentration | | |
|---|---|---|---|
| Treated | treated | Degree of the disease | |
| preparation | (mM) | severity | Control value (%) |
| Exa. 1 | 30 | 5.0 ± 1.0 | 42.3 |
| | 50 | 2.7 ± 1.2 | 69.2 |
| Exa. 2 | 30 | 4 7 ± 0.6 | 46.1 |
| | 50 | 1.3 ± 0.6 | 84.7 |
| Exa. 3 | 30 | 4.7 ± 1.2 | 46.1 |
| | 50 | 0.8 ± 0.3 | 90.4 |
| Exa. 4 | 30 | 5.3 ± 0.6 | 38.5 |
| | 50 | 3.3 ± 1.2 | 61.6 |

| | | |
|---|---|---|
| Non-treated tomatoes | 8.7 ± 0.6 | — |

[Control value (%) is calculated by the formula as described in Table 3.]

As shown in Table 6, when treated with each preparation at the concentrations of 30 and 50 mM, the control values were high (not less than 40% and 60%, respectively).

### TEST EXAMPLE 9

### Controlling Effect Test on Barley Powdery Mildew

Barley(cultivar Dongbori) was planted in the bed soil for horticulture (Bunong Co.), cultivated in a greenhouse for 7 days, and then used in the following experiments Wettable powders prepared from Examples (1)˜(4) were prepared to the concentrations of 30 and 50 mM, respectively, based upon the respective effective ingredients, and then spray-treated on barley leaves and stems using atomizer, respectively, in the amount of 5 ml per pot.

On the other hand, barley infected with powdery mildew was used as an inoculum of the strain Erysiphe graminis causing barley powdery mildew. That is, barley powdery mildew was inoculated and incubated at 20° C. and 60% relative humidity for 10 days while light and darkness were changed at an interval of 12 hours to form spores. 4 hours after spray-treating, the barley infected with barley powdery mildew spores thus formed was inoculated on the spray-treated barley. At this time, the inoculation ratio was 1 pot of the infected barley with barley powdery mildew per 5 pots of the spray-treated barley. The degree of the disease severity was examined in the growth chamber at 20° C. and 60% relative humidity for 10 days while light and darkness were changed at an interval of 12 hours with time. 10 days after the inoculation, the degree of the disease severity was examined based upon the lesion type, lesion size and the ratio of diseased area (see, Test Example 5(A)). The results are shown in Table 7 (mean value±standard deviation).

TABLE 7

| | Concentration | | |
|---|---|---|---|
| Treated | treated | Degree of the disease | |
| preparation | (mM) | severity | Control value (%) |
| Example 1 | 30 | 2.7 ± 0.6 | 55.5 |
| | 50 | 1.2 ± 0.3 | 80.5 |
| Example 2 | 30 | 2.2 ± 0.3 | 63.8 |
| | 50 | 1.2 ± 0.3 | 90.5 |
| Example 3 | 30 | 1.8 ± 0.3 | 69.5 |
| | 50 | 1.0 ± 0.5 | 83.3 |
| Example 4 | 30 | 2.7 ± 0.6 | 55.5 |
| | 50 | 1.7 ± 0.6 | 70.2 |

| | | |
|---|---|---|
| Non-treated barleys | 6.0 ± 0.0 | — |

[Control value(%) is calculated by the formula as described in Table 3.]

As shown in Table 7, when treated with each preparation at the concentrations of 30 and 50 mM, the control values were as high as 55˜90%.

### TEST EXAMPLE 10

### Controlling Effect Test on Wheat Leaf Rust

Wheat(cultivar Eunpa) was planted in the bed soil for horticulture (Bunong Co.), cultivated in a greenhouse for 7 days, and then used in the following experiments. Wettable powder prepared from Examples (1)˜(4) were prepared to the concentrations of 30 and 50 mM, respectively, based upon the respective effective ingredients, and then spray-treated on wheat leaves and stems using atomizer, respectively, in the amount of 5 ml per pot.

On the other hand, wheat infected with wheat leaf rust was used as an inoculum of the strain Puiccina recondita causing wheat leaf rust. That is, wheat leaf rust fungi -were inoculated and incubated at 20° C. and 60% relative humidity for 10 days while light and darkness were changed at an interval of 12 hours to form spores. The spores thus formed were harvested from wheat infected with wheat leaf rust, and then Tween 80 solution at the concentration of 250 ppm(v/v) was added thereto. The resulting solution was titrated using hemacytometer to bring the final concentration to 5×10⁶ spores/ml. 4 hours after spray-treating, the titrated spores were spray-inoculated on the spray-treated wheat in the amounts of 5 ml per a pot. The degree of the disease severity was examined in the growth chamber at 20° C. and 60% relative humidity for 10 days while light and darkness were changed at an interval of 12 hours with time. 10 days after the inoculation, the degree of the disease severity was examined based upon the lesion type, lesion size and the ratio of diseased area (see, Test Example 5(A)). The results are shown in Table 8 (mean value±standard deviation).

TABLE 8

| | Concentration | | |
|---|---|---|---|
| Treated | treated | Degree of the disease | |
| preparation | (mM) | severity | Control value (%) |
| Exa. 1 | 30 | 3.7 ± 0.6 | 52.2 |
| | 50 | 2.7 ± 0.6 | 65.2 |
| Exa. 2 | 30 | 3.5 ± 0.5 | 54.5 |
| | 50 | 2.7 ± 1.2 | 65.2 |
| Exa. 3 | 30 | 3.2 ± 0.8 | 58.7 |
| | 50 | 1.5 ± 0.5 | 80.4 |
| Exa. 4 | 30 | 4.7 ± 0.6 | 39.1 |
| | 50 | 3.7 ± 0.8 | 52.2 |

| | | |
|---|---|---|
| Non-treated wheat | 7.7 ± 0.6 | — |

[Control value(%) is calculated by the formula as described in Table 3.]

za

As shown in Table 8, when treated with each preparation at the concentrations of 30 and 50 mM, the control values were as high as 40˜60% and 50˜80%, respectively.

### Industrial Applicability

The compositions for controlling plant diseases according to the present invention are effective for controlling plant diseases by inducing the expression of defense genes against plant pathogens and bacteria in the early stages of infection without any toxicity, when treated on plant, seeds or its habitat with the compositions. Therefore, the compositions according to the present invention are very useful in the crop protection industries.

What is claimed are:

1. A composition for controlling plant diseases comprising vitamin B1, or its salts or derivatives thereof as an effective ingredient.

2. The composition according to claim 1, wherein the effective ingredient is one or more selected from the group consisting of vitamin B1 hydrochloride, vitamin B1 mononitrate, vitamin B1 monophosphate chloride and vitamin B1 pyrophosphate chloride.

3. The composition according to claim 1, wherein the effective ingredient is present in the range of 5˜100 mM.

4. The composition according to claim 1, wherein the plant is monocotyledonous plant, dicotyledonous plant or seeds thereof.

5. The composition according to claim 1, wherein the plant disease is rice blast, rice bacterial leaf blight, and late blight, powdery mildew or rust of crops

6. The composition according to claim 1, which further comprises other synthetic pesticides, biological pesticides or fertilizers.

7. A method for controlling plant diseases comprising applying the plant, seeds or its habitat to the composition according to any of claims 1 to 6.
